(19) United States
(12) Patent Application Publication
(10) Pub. No.: US 2006/0205788 A1
(43) Pub. Date: Sep. 14, 2006

(54) Compounds having activity at 5ht2c receptor and uses thereof

(76) Inventors: Federica Damiani, Verona (IT); Dieter Hamprecht, Verona (IT); Fabrizio Micheli, Verona (IT); Alessandra Pasquarello, Verona (IT); Giovanna Tedesco, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/548,118

(22) PCT Filed: Feb. 24, 2004
(86) PCT No.: PCT/EP04/01843

§ 371 Date: May 18, 2006

(30) Foreign Application Priority Data
Mar. 5, 2003 (GB) 0305024.2

(51) Int. Cl. A61K 31/454; C07D 403/02

(52) U.S. Cl. 514/326; 546/208

(57) ABSTRACT

Compounds of formula (I) and pharmaceutically acceptable salts thereof are disclosed: 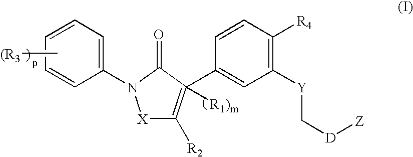
wherein R₁ is hydrogen, fluoro, chloro, hydroxy, C₁₋₆alkyl, C₃₋₇cycloalkyl, C₃₋₇cycloalkyloxy, C₁₋₆alkoxy or haloC₁₋₆alkoxy; m is 0 when ═ is a double bond and m is 1 when ═ is a single bond; R₂ is hydrogen, halogen, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group; X is —(CH₂—CH₂)—, —(CH═CH)—, —(CH₂)₃—, —C(CH₃)₂—, —(CH═CH—CH₂)—, —(CH₂-CH═CH)— or a group —(CHR₅)— wherein R₅ is hydrogen, halogen, hydroxy, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy or C₁₋₆alkylthio; R₃ is halogen, cyano, C₁₋₆alkyl, C₃₋₇cycloalkyl, C₃₋₇cycloalkyloxy, C₁₋₆alkoxy, C₁₋₆alkylthio, hydroxy, amino, mono- or di-C₁₋₆alkylamino, an N-linked 4 to 7 membered heterocyclic group, nitro, haloC₁₋₆alkyl, haloC₁₋₆alkoxy, aryl, arylC₁₋₆alkyl, arylC₁₋₆alkyloxy, arylC₁₋₆alkylthio or COOR₆, CONR₇R₈ or COR₉ wherein R₆, R₇, R₈ and R₉ are independently hydrogen or C₁₋₆alkyl; p is 0, 1 or 2 or 3; R₄ is hydrogen, halogen, hydroxy, cyano, nitro, C₁₋₆alkyl, C₁₋₆alkanoyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group; Y is oxygen, sulfur, —CH₂— or NR₁₀ wherein R₁₀ is hydrogen or C₁₋₆alkyl; D is a single bond, —CH₂—, —(CH₂)₂— or —CH═CH—; and Z is —NR₁₁R₁₂ where R₁₁ and R₁₂ are independently hydrogen or C₁₋₆alkyl, or an optionally substituted N-linked or C-linked 4 to 7 membered heterocyclic group. Method of preparation and uses of the compounds in therapy, for example depression and anxiety, are also disclosed.

This invention relates to novel compounds having pharmacological activity, processes for their preparation, to compositions containing them and to their use in the treatment of CNS and other disorders.

WO 96/23783, WO 97/46699 and WO 97/48700 all disclose a series of indoline derivatives which are 5-HT_{2C} receptor antagonists and which are claimed to be useful in the treatment of various CNS disorders.

A novel class of compounds possessing 5-HT_{2C} receptor activity has been found. The present invention therefore provides, in a first aspect, a compound of formula (I) or a pharmaceutically acceptable salt thereof: 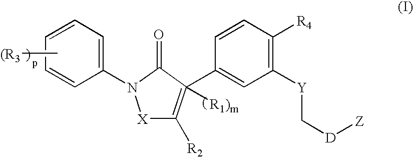
wherein:

R₁ is hydrogen, fluoro, chloro, hydroxy, C₁₋₆alkyl, C₃₋₇cycloalkyl, C₃₋₇cycloalkyloxy, C₁₋₆alkoxy or haloC₁₋₆alkoxy;

m is 0 when ═ is a double bond and m is 1 when ═ is a single bond;

R₂ is hydrogen, halogen, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group;

X is —(CH₂—CH₂)—, —(CH═CH)—, —(CH₂)₃—, —C(CH₃)₂—, —(CH═CH—CH₂)—, —(CH₂—CH═CH)— or a group —(CHR₅)— wherein R₅ is hydrogen, halogen, hydroxy, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy or C₁₋₆alkylthio;

R₃ is halogen, cyano, C₁₋₆alkyl, C₃₋₇cycloalkyl, C₃₋₇cycloalkyloxy, C₁₋₆alkoxy, C₁₋₆alkylthio, hydroxy, amino, mono- or di-C₁₋₆alkylamino, an N-linked 4 to 7 membered heterocyclic group, nitro, haloC₁₋₆alkyl, haloC₁₋₆alkoxy, aryl, arylC₁₋₆alkyl, arylC₁₋₆alkyloxy, arylC₁₋₆alkylthio or COOR₆, CONR₇R₈ or COR₉ wherein R₆, R₇, R₈ and R₉ are independently hydrogen or C₁₋₆alkyl;

p is 0, 1 or 2 or 3;

R₄ is hydrogen, halogen, hydroxy, cyano, nitro, C₁₋₆alkyl, C₁₋₆alkanoyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group;

Y is oxygen, sulfur, —CH₂— or NR₁₀ wherein R₁₀ is hydrogen or C₁₋₆alkyl;

D is a single bond, —CH₂—, —(CH₂)₂— or —CH═CH—; and

Z is —NR₁₁R₁₂ where R₁₁ and R₁₂ are independently hydrogen or C₁₋₆alkyl, or an optionally substituted N-linked or C-linked 4 to 7 membered heterocyclic group.

The following terms, whether used alone or as part of another group are to be given the following meanings, unless otherwise stated.

The term “halogen” and its abbreviated form “halo” are used herein to describe fluorine, chlorine, bromine or iodine.

The term “alkyl” is used herein to describe a straight chain or branched fully saturated hydrocarbon group. “C₁₋₆alkyl” refers to alkyl groups having from one to six carbon atoms, including all isomeric forms, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “C₁₋₆alkanoyl” refers to an alkanoyl group having from 1 to 6 carbon atoms, such as methanoyl (or “formyl”), ethanoyl (or “acetyl”), propanoyl, isopropanoyl, butanoyl, isobutanoyl, sec-butanoyl, pentanoyl, neopentanoyl, sec-pentanoyl, isopentanoyl, tertpentanoyl and hexanoyl.

The term “C₁₋₆alkoxy” refers to a straight chain or branched chain alkoxy (or “alkyloxy”) group having from one to six carbon atoms, including all isomeric forms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

The term “C₃₋₇cycloalkyl” refers to a cycloalkyl group consisting of from 3 to 7 carbon atoms, such as cyclopropane, cyclobutane, cyclopentane, cyclohexane and cycloheptane. Optional substituents for C₃₋₇cycloalkyl includes one or more halogen, hydroxy, oxo, C₁₋₆alkyl, cyano, CF₃, OCF₃, C₁₋₆alkoxy and C₁₋₆alkanoyl.

The term “C₁₋₆alkylthio” refers to a straight chain or branched chain alkylthio group having from one to six carbon atoms, such as methylthio, ethylthio, propylthio, isopropylthio, butylthio, isobutylthio, sec-butylthio, tert-butylthio, pentylthio, neopentylthio, sec-pentylthio, n-pentylthio, isopentylthio, tert-pentylthio and hexylthio.

The term “mono- or di-C₁₋₆alkylamino” refers to an amino group which is substituted by one C₁₋₆alkyl group or an amino group which is substituted by two C₁₋₆alkyl groups, the two amino groups being the same or different. Examples of monoC₁₋₆alkylamino include methylamine, ethylamine, propylamine, isopropylamine, butylamine, isobutylamine, sec-butylamine, tert-butylamine, pentylamine, neopentylamine, sec-pentylamine, n-pentylamine, isopentylamine, tert-pentylamine and hexylamine. Examples of di-C₁₋₆alkylamino include dimethylamine, diethylamine, dipropylamine, diisopropylamine, dibutylamine, diisobutylamine, disec-butylamine, ditert-butylamine, dipentylamine, dineopentylamine, dihexylamine, butylmethylamino, isopropylmethylamino, ethylisopropylamino, ethylmethylamino, etc.

The term “aryl” is used herein to describe groups such as phenyl or naphthyl, which may be optionally substituted by one or more of C₁₋₆alkyl (to form “arylC₁₋₆alkyl”), halogen, CF₃ or C₁₋₆alkoxy (to form “arylC₁₋₆alkoxy”).

The terms “halo C₁₋₆alkoxy” or “haloC₁₋₆alkyl” are used to describe a C₁₋₆alkoxy or a C₁₋₆alkyl group, respectively, substituted with one or more halogens. Examples include —CHCl₂, —CF₃, —OCF₃, etc.

The term ” heterocyclic group” is used herein to describe a stable aromatic or non-aromatic ring containing 1, 2 or 3 heteroatoms selected from nitrogen, sulphur and oxygen. Suitable examples of 4 to 7 membered heterocyclic groups include azetidinyl, pyrrolidinyl, imidazolidinyl, pyrazolidinyl, oxazolinyl, isothiazolidinyl, thiazolidinyl, pyrrolyl, pyrrolinyl, pyrazolinyl, imidazolyl, pyrazolyl, isothiazolyl, thiazolyl, piperidyl, piperazinyl, morpholinyl, pyridyl, pyridazinyl, pyrimidinyl, pyrazinyl, azepinyl, azepanyl, dioxolanyl, thienyl, tetrahydrothienyl, tetrahydrofuryl, dioxanyl and dithianyl.

The term “N-linked heterocyclic group” is used herein to describe a heterocyclic groupwhich is linked to the remainder of the molecule via a nitrogen atom. Suitable examples of 4 to 7 membered N-linked heterocyclic groups include azetidinyl, pyrrolidinyl, imidazolidinyl, pyrazolidinyl, oxazolinyl, isothiazolidinyl, thiazolidinyl, pyrrolyl, pyrrolinyl, pyrazolinyl, imidazolyl, pyrazolyl, piperidyl, piperazinyl, morpholinyl and azepanyl.

The term “C-linked heterocyclic group” is used herein to describe a heterocyclic group which is linked to the remainder of the molecule via a carbon atom. Suitable examples of 4 to 7 membered C-linked heterocyclic groups include azetidinyl, pyrrolidinyl, imidazolidinyl, pyrazolidinyl, oxazolinyl, isothiazolidinyl, thiazolidinyl, pyrrolyl, pyrrolinyl, pyrazolinyl, imidazolyl, pyrazolyl, isothiazolyl, thiazolyl, piperidyl, piperazinyl, morpholinyl, pyridyl, pyridazinyl, pyrimidinyl, pyrazinyl, azepinyl, azepanyl, dioxolanyl, thienyl, tetrahydrothienyl, tetrahyrdofuryl, dioxanyl and dithianyl.

More than one optional substituent may be present in the N-linked or C-linked heterocycle, which may be the same or different, and may be attached to any carbon atom of the heterocycle or an available nitrogen atom.

Suitable optional substituents for the N-linked or C-linked heterocycle include C₁₋₆alkyl, amino, mono- or di-C₁₋₆alkylamino, aryl, arylC₁₋₆alkyl, arylamino, hydroxy, C₁₋₆alkylamido, hydroxyC₁₋₆alkyl, C₁₋₆alkoxycarbonyl, halogen, haloC₁₋₆alkyl, a heteroaromatic group (such as indole or benzimidazole), an aromatic or non-aromatic N-linked or C-linked heterocycle or an aromatic or non-aromatic heterocycleC₁₋₆alkyl optionally substituted by C₁₋₆alkyl. Examples of aromatic or non-aromatic heterocycleC₁₋₆alkyl include heterocycle-methyl (such as pyridinyl-methyl and benzimidazolyl-methyl) and heterocycle-ethyl (such as morpholinyl-ethyl and indolyl-ethyl).

Substituents in the N-linked or C-linked heterocycle may form a bridge structure, to form a group such as for example 2-oxa-5-azabicyclo[2.2.1]heptyl. Such a bicyclic group may be further substituted by the substituents listed above. More than one substituent may be present on the same carbon atom to form spiro structures such as 1,4 and 1,5 dioxa spiro compounds.

When X is a group —(CHR₅)—, preferably R₅ is hydrogen. Preferably X is —CH₂—.

When ═ is a single bond, preferably R₁ is hydrogen, hydroxy or C₁₋₆alkoxy.

Preferably R₂ is hydrogen.

When p is 2 or 3, R₃ may be the same or different. Preferably p is 1 or 2 and R₃ is/are halogen, particularly chloro or fluoro, attached at the 3 or the 3,4-positions of the phenyl ring.

Preferably R₄ is C₁₋₆alkoxy (particularly methoxy), OCF₃, halogen or cyano.

Preferably Y is oxygen.

Preferably D is —CH₂—.

Preferably Z is an optionally substituted N-linked 4 to 7 membered heterocycle, in particular piperidyl. Preferred substituents include halogen (particularly fluoro) and C₁₋₆alkyl (particularly methyl).

Preferred compounds are compounds of formula (Ia):

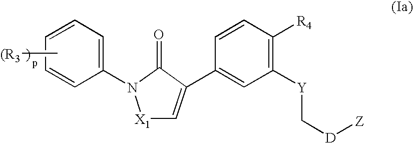

wherein R₃, p, R₄, Y, D, Z, ═ are as defined for formula (I) and X₁ is —CH₂— or —HC(OH)—. Preferred features of formula (I) also apply to formula (Ia).

Preferred compounds include:
1. 1-(3,4-Dichloro-phenyl)-5-hydroxy-3-[4-methoxy-3-(2-piperidin-1-yl-ethoxy)-phenyl]-pyrrolidin-2-one
2. 1-(3,4-Dichloro-phenyl)-3-[4-methoxy-3-(2-piperidin-1-yl-ethoxy)-phenyl]-1,5-dihydro-pyrrole-2-one
3. 1-(3,4-Dichloro-phenyl)-3-[4-methoxy-3-(2-piperidin-1-yl-ethoxy)-phenyl]-pyrrolidin-2-one
4. 1-(3,4-Dichloro-phenyl)-3-[4-methoxy-3-(2-piperidin-1-yl-ethoxy)-phenyl]-3-methyl-pyrrolidin-2-one
6. 1-(4-Methoxyphenyl)-3-[4-methoxy-3-(2-piperidin-1-yl-ethoxy)-phenyl]-1,5-dihydro-pyrrole-2-one
7. 1-(4-Methoxyphenyl)-3-[4-methoxy-3-(2-piperidin-1-yl-ethoxy)-phenyl]-pyrrolidin-2-one
8. 1-(3-Chloro-4-methoxy-phenyl)-3-[4-methoxy-3-(2-piperidin-1-yl-ethoxy)-phenyl]-pyrrolidin-2-one
9. 1-(3-Chloro-4-methoxy-phenyl)-3-[4-methoxy-3-(2-piperidin-1-yl-ethoxy)-phenyl]-1,5-dihydro-pyrrole-2-one
10. 1-(3-Fluoro-4-methoxy-phenyl)-3-[4-methoxy-3-(2-piperidin-1-yl-ethoxy)-phenyl]-pyrrolidin-2-one
11. 1-(3-Fluoro-4-methoxy-phenyl)-3-[4-methoxy-3-(2-piperidin-1-yl-ethoxy)-phenyl]-1,5-dihydro-pyrrole-2-one
12. 1-(3-Fluoro-phenyl)-3-[4-methoxy-3-(2-piperidin-1-yl-ethoxy)-phenyl]-pyrrolidin-2-one
13. 1-(3-Fluoro-phenyl)-3-[4-methoxy-3-(2-piperidin-1-yl-ethoxy)-phenyl]-1,5-dihydro-pyrrole-2-one
14. 1-(3,4-Dichloro-phenyl)-5,5-dimethyl-4-hydroxy-3-[4-methoxy-3-(2-piperidin-1-yl-ethoxy)-phenyl]-pyrrol-2-one
15. 1-(3,4-Dichloro-phenyl)-5,5-dimethyl-4-hydroxy-3-[4-methoxy-3-(2-piperidin-1-yl-ethoxy)-phenyl]-pyrrol-2-one

and pharmaceutically acceptable salts thereof.

The compounds of formula (I) can form acid addition salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid.

The compounds of this invention may be in crystalline or non-crystalline form, and, if crystalline, may optionally be hydrated or solvated. This invention includes within its scope stoichiometric hydrates as well as compounds containing variable amounts of water.

Certain compounds of formula (I) are capable of existing in stereoisomeric forms (e.g. geometric or (“cis-trans”) isomers, diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (I) or a pharmaceutically acceptable salt thereof, which process comprises:

(a) reacting a compound of formula (II):

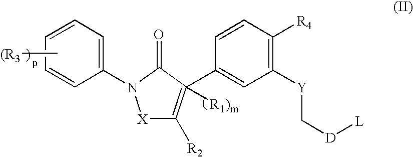

wherein R₁, R₂, R₃, R₄, m, p, X, ═, Y and D are as defined for formula (I), and L is a leaving group, with a compound of formula (III):
Z-H (III)

wherein Z is as defined for formula (I); or

(b) cyclising a compound of formula (IV):

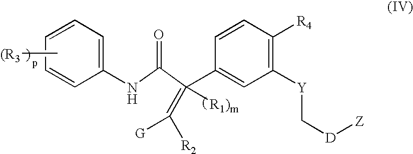

wherein R₁, R₂, m, R₃, p, R4, Y, D, Z and ═ are as defined for formula (I) and G is a group —X═CH₂, wherein X is as defined for formula (I), dehydrogenated as required;

and thereafter, for either process (a) or process (b), optionally followed by:

removing any protecting groups; and/or

converting a compound of formula (I) into another compound of formula (I); and/or

forming a pharmaceutically acceptable salt.

For the reaction of process (a), suitably L is mesylate. The reaction may take place in a solvent such as DMF in the presence of sodium iodide and potassium carbonate.

The reaction of process (b) suitably takes place in a solvent such as THF in the presence of OSO₄ and NaIO₄.

Compounds of formula (I) can be converted into further compounds of formula (I) using standard techniques. For example, and by way of illustration rather than limitation, a compound wherein X is —(HCOH)— may be converted to a compound wherein X is —(CH₂)— by using a suitable reducing agent such as triethylsilane-trifluoroacetic acid using dichloromethane as solvent, and a compound wherein R₁ is hydroxy may be converted to compound wherein m is 0 and ═ is a double bond by an elimination reaction in TFA.

Compounds of formulae (II), (III) and (IV) are commercially available or may be prepared according to methods described herein or may be prepared according to known methods or by analogous methods thereto.

Those skilled in the art will appreciate that it may be necessary to protect certain groups to carry out the above processes. Suitable protecting groups and methods for their attachment and removal are conventional in the art of organic chemistry, such as those described in Greene T. W. ‘Protective groups in organic synthesis’ New York, Wiley (1981).

Pharmaceutically acceptable salts may be prepared conventionally by reaction with the appropriate acid or acid derivative.

In another aspect, the present invention provides a pharmaceutical composition comprising a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, Is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose);, fillers (e.g. lactose, microcrystalline cellulose or calcium hydrogen phosphate); tabletting lubricants lubricants (e.g. magnesium stearate, talc or silica);, disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The compounds of the present invention have affinity for the 5-HT_{2C} receptor. The affinity can be determined by assessing their ability to displace [³H]-mesulergine from rat or human 5-HT_{2C} clones expressed in 293 cells in vitro, as described in WO 94/04533.

All the Example compounds were tested according to this assay and were found to have pKi values >5.8. Some compounds show a considerably higher affinity in the range of 7.0 to >9.0 in human cells.

The intrinsic activity of the compounds of this invention can be determined according to the [³⁵S]GTPγS functional assay which is described in WO 99/07700.

Compounds of formula (I) and their pharmaceutically acceptable salts are of use in the treatment of certain CNS disorders such as depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, vascular dementia with depressed mood, schizoaffective disorder or the depressed type, and depressive disorders resulting from general medical conditions including, but not limited to, myocardial infarction, diabetes, miscarriage or abortion, etc), anxiety including generalised anxiety and social anxiety disorder, schizophrenia, panic disorder, agoraphobia, social phobia, epilepsy, obsessive compulsive disorder and post-traumatic stress disorder, pain (particularly neuropathic pain), migraine, memory disorders, including dementia, amnesic disorders and age-associated memory impairment, disorders of eating behaviours including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), sedative ipnotic, amphetamine or amphetamine-related drugs (e.g. dextroamphetamine, methylamphetamine) or a combination thereof, Alzheimer's disease, motor disorders such as Parkinson's disease, dementia in Parkinson's disease, neuroleptic-induced Parkinsonism and tardive dyskinesias, as well as other psychiatric disorders, disorders associated with spinal trauma and/or head injury such as hydrocephalus, gastrointestinal disorders such as IBS (Irritable Bowel Syndrome), Crohn's disease, ulcerative colitis, non-steroidal anti-inflammatory drug induced damage) as well as microvascular diseases such as macular oedema and retinopathy.

It is to be understood that, as used herein, the term “treatment” refers to alleviation of established symptoms as well as prophylaxis.

Thus the present invention also provides a compound of formula (I) or a pharmaceutically acceptable salt thereof, for use as a therapeutic substance. In particular, the present invention provides a compound of formula (I) or a pharmaceutically acceptable salt thereof for use in the treatment of the above disorders. In particular the invention provides a compound of formula (I) or a pharmaceutically acceptable salt thereof for use as a therapeutic substance in the treatment of a CNS disorder. Preferably the CNS disorder is depression or anxiety.

Compounds of the invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of the combination or composition may be administered simultaneously (either in the same or different pharmaceutical formulations), separately or sequentially.

The invention further provides a method of treatment of the above disorders in mammals including humans, which comprises administering to the sufferer a therapeutically safe and effective amount of a compound of formula (I) or a pharmaceutically acceptable salt thereof. In particular the invention provides a a method of treatment of a CNS disorder in mammals including humans, which comprises administering to the sufferer a therapeutically safe and effective amount of a compound of formula (I) or a pharmaceutically acceptable salt thereof. Preferably the disorder is depression or anxiety.

In another aspect, the invention provides for the use of a compound of formula (I) or a pharmaceutically acceptable salt thereof in the manufacture of a medicament for use in the treatment of the above disorders. In particular the present invention provides the use of a compound of formula (I) or a pharmaceutically acceptable salt thereof in the manufacture of a medicament for use in the treatment of a CNS disorder. Preferably the CNS disorder is depression or anxiety.

The composition of the present invention may Contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months. When administered in accordance with the invention, no unacceptable toxicological effects are expected with the compounds of the invention.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the present invention.

### Preparation 1: [4-Methoxy-3-(2-piperidin-1-yl-ethoxy)-phenyl]-acetic acid 2-piperidin-1-yl-ethyl ester

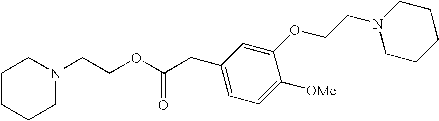

A mixture of (3-hydroxy-4-methoxy-phenyl)-acetic acid (1.85 g), dry DMF (25 ml), K₂CO₃ (5.9 g) and N-chloroethylpiperidine hydrochloride (3.74 g) was heated at 40° C. for 5 h. Volatiles were then removed in vacuo and the residue partitioned between water and EtOAc. The organic layer was washed (brine) and concentrated to give the title (3.76 g) compound as an orange oil.

NMR (¹H, CDCl₃): δ 6.93-6.80 (m, 3H), 4.22 (t, 2H), 4.14 (t, 2H), 3.82 (s, 3H), 3.55 (s, 2H), 2.82 (t, 2H), 2.66-2.40 (m, 10H), 1.66-1.40 (m, 12H).

### Preparation 2: [4-Methoxy-3-(2-piperidin-1-yl-ethoxy)-phenyl]-acetic acid methyl ester

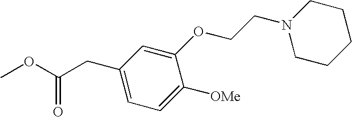

[4-Methoxy-3-(2-piperidin-1-yl-ethoxy)-phenyl]-acetic acid 2-piperidin-1-yl-ethyl ester (6.3 g) in MeOH (6 ml), THF (6 ml) and water (6 ml) containing KOH (1.7 g) was heated at 45° C. for 1 h and then allowed to cool to 25° C. over 90 min. With stirring in an ice bath conc. aqueous HCl (6 ml) was then added. The mixture was evaporated to dryness. The material was heated at reflux with HCl in MeOH (1 M) for 4 h, concentrated and extracted with CH₂Cl₂. The mixture was filtered and the solvent removed in vacuo to give the title compound (4.4 g) as an orange oil.

NMR (¹H, CDCl₃): δ 6.88-6.79 (m, 3H), 4.18 (t, 2H), 3.84 (s, 3H), 3.69 (s, 3H), 3.55 (s, 2H), 2.87 (t, 2H), 2.56 (bs, 4H), 1.66 (bs, 4H), 1.46 (bs, 2H).

### Preparation 3: 2-[4-Methoxy-3-(2-piperidin-1-yl-ethoxy)-phenyl]-pent-4-enoic acid methyl ester

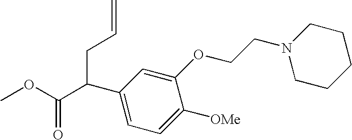

Procedure: To a solution of [4-methoxy-3-(2-piperidin-1-yl-ethoxy)-phenyl]-acetic acid methyl ester (2.1 g) in THF (dry, 15 ml) at −78° C. was slowly added lithium bis(trimethylsilyl)amide (1 M in THF, 8.2 ml). The solution was stirred at this temperature for 15 min before allyl bromide (0.59 ml) was added. After additional 30 min water and EtOAc were added with,stirring. The mixture was allowed to warm to 25° C., layers separated and the organic layer washed (brine), concentrated and submitted to column chromatography (silica gel, CH₂Cl₂/MeOH/NH₃) to give the title compound (1.3 g) as a colourless oil.

NMR (¹H, CDCl₃): δ 6.90-6.80 (m, 3H), 5.76-5.67 (m, 1H), 5.10-4.98 (m, 2H), 4.18 (bs, 2H), 3.83 (s, 3H), 3.65 (s, 3H), 3.56 (t, 1H), 2.86 (bs, 2H), 2.83-2.73 (m, 1H), 2.65-2.45 (m, 5H), 1.65 (bs, 4H), 1.47 (bs, 2H). MS (m/z): 348 [MH]⁺.

### Preparation 4: 2-[4-Methoxy-3-(2-piperidin-1-yl-ethoxy)-phenyl]-pent-4-enoic acid hydrochloride salt

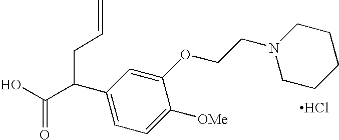

2-[4-Methoxy-3-(2-piperidin-1-yl-ethoxy)-phenyl]-pent-4-enoic acid methyl ester (1.3 g) in MeOH (2 ml), THF (2 ml) and water (2 ml) containing KOH (0.42 g) was heated at 45° C. for 1 h and then allowed to cool to 25° C. The mixture was evaporated to dryness. THF (5 ml) and conc. aqueous HCl (0.62 ml) were added, the mixture concentrated, extracted with CH₂Cl₂, filtered and the solvent removed in vacuo to give the title compound (1.2 g) as an off-white foam.

NMR (¹H, CD₃OD): δ 7.05 (d, 1H), 6.96 (dd, 1H), 6.91 (d, 1H), 5.81-5.70 (m, 1H), 5.02 (dd, 1H), 4.92 (dd, 1H), 4.31 (dd, 2H), 3.83 (s, 3H), 3.48-3.42 (m, 3H), 3.37-3.28 (m, 4H), 2.78-2.69 (m, 1H), 2.44-2.35 (m, 1H), 1.91-1.84 (m, 4H), 1.72-1.63 (m, 2H). MS (m/z): 334 [MH]⁺.

### Preparation 5: 2-[4-Methoxy-3-(2-piperidin-1-yl-ethoxy)-phenyl]-pent-4-enoic acid (3,4-dichlorophenyl)-amide

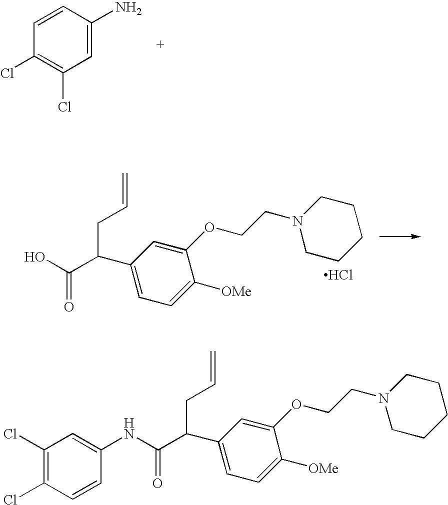

To a solution of 2-[4-methoxy-3-(2-piperidin-1-yl-ethoxy)-phenyl]-pent-4-enoic acid hydrochloride salt (0.46 g) in dry CH₂Cl₂ (4 ml), under N₂, was added, at 0° C., oxalyl chloride (0.11 ml) and DMF (cat). After 30 min the reaction mixture was concentrated to dryness in vacuo. To this material was added toluene (dry, 4 ml) and 3,4-dichloroaniline (0.20 g). The mixture was heated at 105° C. for 4 h, then partitioned between aqueous NaHCO₃ and EtOAc. The organic layer was washed (brine), concentrated and purified by column chromatography (silica gel, CH₂Cl₂/MeOH/NH₃) to give the title compound (0.35 g) as a slightly brown oil.

NMR (¹H, CDCl₃): δ 7.74 (s, 1H), 7.55 (bs, 1H), 7.31 (bs, 2H), 6.96 (bs, 1H), 6.91-6.83 (m, 2H), 5.77-5.68 (m, 1H), 5.09 (d, 1H), 5.01 (d, 1H), 4.23-4.16 (m, 2H), 3.85 (s, 3H), 3.53 (t, 1H), 2.99-2.91 (m, 1H), 2.83 (t, 2H), 2.55 (bs, 4H), 1.68-1.62 (m, 4H), 1.47 (bs, 3H). MS (m/z): 477 [MH]⁺, 2Cl.

### Preparation 6: 2-[4-Methoxy-3-(2-piperidin-1-yl-ethoxy)-phenyl]-propanoic acid methyl ester

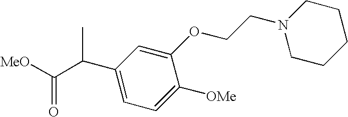

To a solution of [4-methoxy-3-(2-piperidin-1-yl-ethoxy)-phenyl]-acetic acid methyl ester (0.60 g) in THF (dry, 6 ml) at −78° C. was slowly added lithium bis(trimethylsilyl)amide (1 M in THF, 2.3 ml). The solution was stirred at this temperature for 15 min before iodomethane (0.12 ml) was added, then allowed to warm to 25° C. After 16 h aqueous NaHCO₃ and EtOAc were added with stirring, layers separated and the organic layer washed (brine), concentrated and submitted to column chromatography (silica gel, CH₂Cl₂/MeOH/NH₃) to give the title compound (0.27 g) as a slightly yellow oil.

NMR (¹H, CD₃OD): δ 6.88-6.75 (m, 3H), 4.12 (t, 2H), 3.80 (s, 3H), 3.67-3.34 (m, 4H), 2.78 (t, 2H), 2.50 (bs, 4H), 1.65-1.37 (m, 9H). MS (m/z): 322 [MH]⁺.

### Preparation 7: 2-(3,4-Dichloro-phenylamino)-2-methyl-propionic acid

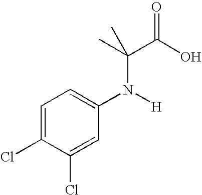

To a solution of 3,4-dichloroaniline (2 g, 12.4 mmol) and 1,1,1-trichloro-2-methyl-2-propanol 0.5 hydrate (3.47 g, 18.63 mmol) in acetone (25 ml) was added KOH (2.79 g, 49.7 mmol) at 0° C., and the mixture was stirred for 1 h at 0° C. and overnight at room temperature. The reaction mixture was concentrated in vacuo, diluted with water, and washed with diethyl ether. The acqueous solution was acidified with citric acid and extracted with AcOEt (3×150 ml). The organic layer was washed with brine, dried over anhydrous NaSO₄ and concentrated in vacuo to give the title compound in 2.2 g yield as a white solid (72%).

NMR (¹H, DMSO): δ 12.30 (bs,1H), 7.18 (d,1H), 6.60 (s, 1H), 6.40 (dd, 1H), 4.98 (bs, 1H), 1.40 (s, 4H).

### Preparation 8: Methyl 2-(3,4-dichloro-phenylamino)-2-methyl-propionate

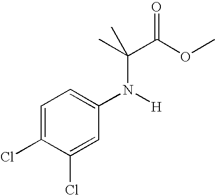

To a solution of 2-methyl-2-(3,4-dichloroaniline)-propionic acid (0.50 g, 2.02 mmol) in MeOH+CH₂Cl₂ (14+7 ml) were added triethylamine (0.57 ml, 4.08 mmol) and Me₃SiCHN₂ (2M in hexane, 6.06 mmol) at 0° C. The reaction mixture was stirred for 4 h at room temperature, concentrated in vacuo and the crude product purified by flash chromatography (AcOEt:cyclohexane=1:9) to give the title compound in 489 mg yield as white solid (93%).

NMR (¹H, DMSO): δ 7.42 (dd,1H), 6.57 (d, 1H), 6.46 (s, 1H), 6.35 (dd, 1H), 3.62 (s, 3H), 1.45 (s, 6H).

### Preparation 9: 2-[4-Methoxy-3-(2-piperidin-1-yl-ethoxy)-phenyl]-acetic acid hydrochloride salt

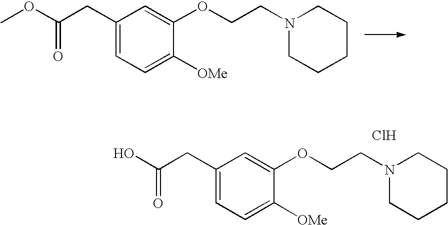

To a solution of methyl-2-(3-[2-piperidin-1-yl-ethoxy]-4-methoxy-phenyl)-acetate (0.59 g, 1.92 mmol) in THF+MeOH (1+1 ml) at 25° C. was added a solution of KOH (0.21 g, 3.84 mmol) in H₂O (1 ml). The solution was heated at 45° C. for 1.5 hours. After removing the solvent in vacuo the residue was dissolved in THF and acidified at 0° C. with conc. aqueous HCl. The mixture was concentrated to dryness in vacuo, the crude was extracted with PrOH/CH₂Cl₂ (1/1), filtered and concentrated to dryness in vacuo to give the title compound in 544 mg yield as brown solid (y=96%). MS (m/z): 294 [MH]⁺.

### Preparation 10: 2-((3,4-Dichloro-phenyl)-{2-[4-methoxy-3-(2-piperidin-1-yl-ethoxy)-phenyl]-acetyl}-amino)-2-methyl propionic acid methyl ester

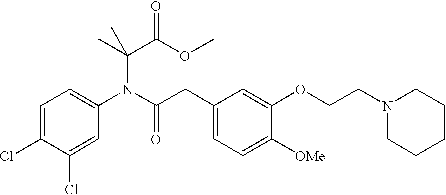

To a solution of 2-(3-[2-piperidin-1-yl-ethoxy]-4-methoxy-phenyl)-acetic acid hydrochloride salt (0.62 g, 1.89 mmol) in CH₂Cl₂ (9 ml) at 0° C. were added oxalyl chloride (0.33 ml, 3.78 mmol) and DMF (some drops). The solution was stirred at room temperature for 1.5 hours and concentrated to dryness in vacuo. The crude was dissolved in 1,4-dioxane, added with a solution of methyl-2-methyl-2-(3,4-dichloroaniline)-propionate (0.47 g, 1.78 mmol) in 1,4-dioxane and heated at 90° C. for 20 hours. The reaction mixture was concentrated in vacuo, dissolved in CH₂Cl₂, washed with conc. aqueous NaHCO₃, brine and dried over anhydrous NaSO₄. After purification by flash chromatography (CH₂Cl₂:MeOH=9:1) the title compound was obtained in 681 mg yield as a brown oil (61%). MS (m/z): 537 [MH]⁺ (2CI).

### EXAMPLE 1

### 1-(3,4-Dichloro-phenyl)-5-hydroxy-3-[4-methoxy-3-(2-piperidin-1-yl-ethoxy)-phenyl]-pyrrolidin-2-one

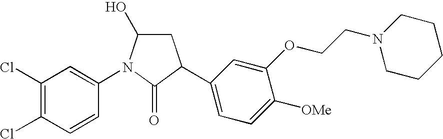

To a solution of 2-[4-methoxy-3-(2-piperidin-1-yl-ethoxy)-phenyl]-pent-4-enoic acid (3,4-dichlorophenyl)-amide (0.30 g) in THF/H₂O (5/1, 12 ml) was added OSO₄ (4% wt in water, 0.2 ml) and NaIO₄ (0.41 g). After 20 hours excess aqueous Na₂S₂O₃ was added with stirring. After 5 min. the mixture was partitioned between sat. aqueous NaHCO₃ and EtOAc. The organic layer was washed (brine), concentrated to dryness in vacuo and the residue purified by column chromatography to give the title product as a colourless foam (0.20 g, ca. 2:1 mixture of diastereoisomers):

NMR (¹H, CDCl₃): δ 7.80 and 7.74 (2d, 1H), 7.55-7.35 (m, 2H), 7.00 (s, 0.33H), 6.92 (d, 0.33H), 6.83-6.70 (m, 2.34H), 5.70-5.59 (m, 1H), 4.17-4.00 (m, 3.67H), 3.82 (s, 3H), 3.70 (dd, 0.33H), 2.95-2.85 (m, 0.33H), 2.77 (t, 2H), 2.55-2.34 (m, 5.34H), 2.17-2.08 (m, 0.33H), 1.62-1.50 (m, 4H), 1.46-1.35 (m, 2H). MS (m/z): 479 [MH]⁺, 2Cl.

### EXAMPLE 2

### 1-(3,4-Dichloro-phenyl)-3-[4-methoxy-3-(2-piperidin-1-yl-ethoxy)-phenyl]-1,5-dihydro-pyrrole-2-one

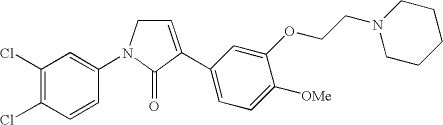

To 1-(3,4-dichloro-phenyl)-5-hydroxy-3-[4-methoxy-3-(2-piperidin-1-yl-ethoxy)-phenyl]-pyrrolidin-2-one (0.13 g) in CH₂Cl₂ (dry, 5 ml) and Et₃SiH (0.13 ml) at 0° C. was added dropwise a solution of trifluoroacetic acid (0.22 ml) in CH₂Cl₂ (dry, 1 ml). The mixture was allowed to warm to 25° C. After 20 h volatiles were removed in vacuo and the residue submitted to column chromatography (silica gel, CH₂Cl₂/MeOH/NH₃) to give a mixture of 1-(3,4-dichloro-phenyl)-3-[4-methoxy-3-(2-piperidin-1-yl-ethoxy)-phenyl]-pyrrolidin-2-one and 1-(3,4-dichloro-phenyl)-3-[4methoxy-3-(2-piperidin-1-yl-ethoxy)-phenyl]-1,5-dihydro-pyrrole-2-one. On trituration with EtOAc : petroleum ether (40-60) 1:2 the title compound was obtained as an off-white solid.

NMR (¹H, CDCl₃): δ 7.99 (d, 1H), 7.70 (dd, 1H), 7.54-7.50 (m, 2H), 7.46 (d, 1H), 7.22 (t, 1H), 6.92 (d, 1H), 4.43 (d, 2H), 4.23 (t, 2H), 3.91 (s, 3H), 2.87 (t, 2H), 2.56 (bs, 4H), 1.70-1.60 (m, 4H), 1.47 (bs, 2H). MS (m/z): 461 [MH]⁺, 2Cl. mp: 98-99° C.

### EXAMPLE 3

### 1-(3,4-Dichloro-phenyl)-3-[4-methoxy-3-(2-piperidin-1-yl-ethoxy)-phenyl]-pyrrolidin-2-one

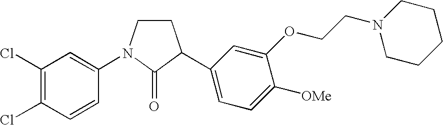

The title compound (22 mg) was obtained as a colourless film from the mother liquor from Example 2 by removal of the volatiles, extraction with hexane and concentration of the hexane solubles in vacuo.

NMR (¹H, CDCl₃): δ 7.87 (d, 1H), 7.60 (dd, 1H), 7.43 (d, 1H), 6.89-6.83 (m, 3H), 4.17 (t, 2H), 3.95-3.80 (m, 6H), 2.82 (t, 2H), 2.70-2.60 (m, 1H), 2.52 (bs, 4H), 2.35-2.25 (m, 1H), 1.64-1.58 (m, 4H), 1.50-1.42 (m, 2H). MS (m/z): 463 [MH]⁺, 2Cl.

### EXAMPLE 4

### 1-(3,4-Dichloro-phenyl)-5-methoxy-3-[4-methoxy-3-(2-piperidin-1-yl-ethoxy)-phenyl]-pyrrolidin-2-one

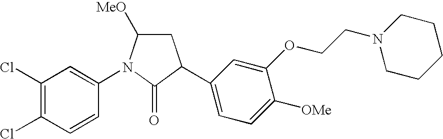

1-(3,4-Dichloro-phenyl)-5-hydroxy-3-[4-methoxy-3-(2-piperidin-1-yl-ethoxy)-phenyl]-pyrrolidin-2-one was treated with MeOH (dry, 2 ml) containing pyridinium 4-toluenesulfonate (42 mg) at 25° C. for 4 days, at 50° C. for 4 h and at 60° C. for 6 h. The mixture was partitioned between aqueous NaHCO₃ and EtOAc. The organic layer was collected, concentrated and submitted to column chromatography (silica gel, CH₂Cl₂/MeOH/NH₃). A major diastereoisomer (3,5-trans substitution of the pyrrolidin-2-one, 25 mg slightly yellow film) was isolated besides a ca. 1:1 mixture of the major and the minor diastereoisomer (4 mg colourless film) and recovered unreacted starting material (21 mg 1-(3,4-dichloro-phenyl)-5-hydroxy-3-[4-methoxy-3-(2-piperidin-1-yl-ethoxy)-phenyl]-pyrrolidin-2-one).

Major diastereoisomer: NMR (¹H, CDCl₃): δ 7.81 (d, 1H), 7.51 (d, 1H), 7.45 (d, 1H), 6.90-6.82 (m, 3H), 5.32 (d, 1H), 4.17 (t, 2H), 4.02 (dd, 1H), 3.85 (s, 3H), 3.41 (s, 3H), 2.84 (t, 2H), 2.64 (dd, 1H), 2.56 (bs, 4H), 2.37-2.30 (m, 1H), 1.67-1.60 (m, 4H), 1.50-1.40 (m, 2H). MS (m/z): 493 [MH]⁺, 2Cl.

Ca. 1:1 mixture of major and minor diastereoisomer: NMR (¹H, CDCl₃): δ 7.82 and 7.74 (2d, 1H), 7.51 (d, 0.5H), 7.48-7.42 (m, 1H), 7.03 (d, 0.5H), 6.97 (dd, 0.5 H), 6.91-6.83 (m, 2.5H), 5.37-5.33 (m, 1H), 4.26-4.20 (m, 2H), 4.02 (dd, 0.5H), 3.85 (2s, 3H), 3.77 (dd, 0.5H), 3.41 and 3.38 (2s, 3H), 2.89 (bs, 2H), 2.85-2.78 (m, 0.5H), 2.70-2.50 (m, 4.5H), 2.37-2.24 (m, 1H), 1.66 (bs, 4H), 1.48 (bs, 2H). MS (m/z): 493 [MH]⁺, 2Cl.

### EXAMPLE 5

### 1-(3,4-Dichloro-phenyl)-3-[4-methoxy-3-(2-piperidin-1-yl-ethoxy)-phenyl]-3-methyl-pyrrolidin-2-one

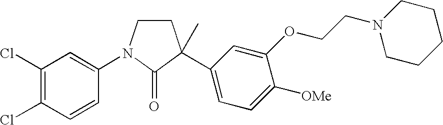

To a solution of 2-[4-Methoxy-3-(2-piperidin-1-yl-ethoxy)-phenyl]-propanoic acid methyl ester (0.27 g) in THF (dry, 5 ml) at −78° C. was slowly added lithium bis(trimethylsilyl)amide (1 M in THF, 1.0 ml). The solution was stirred at this temperature for 15 min before allyl bromide (0.11 ml) was added. The mixture was allowed to warm to 0° C. and kept at that temperature for 1.5 h. It was then partitioned between aqueous NaHCO₃ and EtOAc, layers separated and the organic layer washed (brine), concentrated and submitted to column chromatography (silica gel, CH₂Cl₂/MeOH/NH₃) to give a colourless oil (0.14 g).

This material in MeOH (1 ml) containing KOH (5 eq.) was heated at 45° C. for 3 h and then and then kept at 25° C. for 18 h. The mixture was evaporated to dryness. Excess aqueous HCl was added, the mixture concentrated, extracted with CH₂Cl₂, filtered and the solvent removed in vacuo to give a colourless foam (0.09 9).

To this material in dry CH₂Cl₂ (1 ml), under N₂, was added, at 0° C., oxalyl chloride (0.03 ml) and DMF (cat). After 1 h the reaction mixture was concentrated to dryness in vacuo.

To this material was added dioxane (dry, 1 ml) and 3,4-dichloroaniline (0.04 g). The mixture was heated at 95° C. for 4 h, concentrated in vacuo and purified by column chromatography (silica gel, CH₂Cl₂/MeOH/NH₃) to give an orange oil (0.06 g).

This material was converted to 1-(3,4-dichloro-phenyl)-5-hydroxy-3-methyl-3-[4-methoxy-3-(2-piperidin-1-yl-ethoxy)-phenyl]-pyrrolidin-2-one (37 mg colourless film) in analogy to the procedure described for Example 1.

To 1-(3,4-dichloro-phenyl)-5-hydroxy-3-methyl-3-[4-methoxy-3-(2-piperidin-1-yl-ethoxy)-phenyl]-pyrrolidin-2-one (30 mg) in CH₂Cl₂ (dry, 0.5 ml) and Et₃SiH (0.049 ml) was added dropwise a solution of trifluoroacetic acid (0.047 ml) in CH₂Cl₂ (dry, 0.5 ml) at 0° C. The mixture was allowed to warm to 25° C. After 7.5 h volatiles were removed in vacuo and the residue submitted to column chromatography (silica gel, CH₂Cl₂/MeOH/NH₃) to give the title compound (19 mg) as a colourless film.

NMR (¹H, CDCl₃): δ 7.88 (d, 1H), 7.57 (dd, 1H), 7.41 (d, 1H), 7.01 (d, 1H), 6.92 (dd, 1H), 6.82 (d, 1H), 4.14 (t, 2H), 3.84 (s, 3H), 3.75-3.66 (m, 2H), 2.79 (t, 2H), 2.64-2.57 (m, 1H), 2.58 (bs, 4H), 2.29-2.21 (m, 1H), 1.65-1.55 (m, 7H), 1.50-1.40 (m, 2H). MS (m/z): 477 [MH]⁺, 2Cl.

### EXAMPLE 6

### 1-(4-Methoxyphenyl)-3-[4-methoxy-3-(2-piperidin-1-yl-ethoxy)-phenyl]-1,5-dihydro-pyrrole-2-one hydrochloride

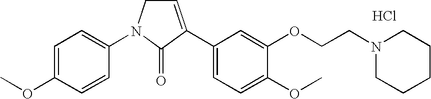

To a solution of 2-[4-methoxy-3-(2-piperidin-1-yl-ethoxy)-phenyl]-pent-4-enoic acid (4-methoxyphenyl)-amide (prepared in an analogous way to Procedure 5, 570 mg, 1.3 mmol) in acetone/H₂O 8/1 (33 ml) was added N-methyl-morpholine-N-oxide (2eq, 304 mg) and OSO₄ (4wt % sol. in water, cat., 0.84 ml). The reaction was stirred at room temperature for 6 hours and then quenched with 40 ml of Na₂SO₃ sat. After 15 minutes stirring the diol was extracted with ethyl acetate (2×20 ml), dried over Na₂SO₄, filtered and concentrated to dryness in vacuo.

The crude product was then dissolved in THF/H₂O 1/1 (30 ml) and potassium periodate (1.5 eq, 391 mg) was added. The reaction mixture was stirred at room temperature for 3 hours. The solution was diluted with water (20 ml) and extracted with ethyl acetate (3×20 ml). The combined organic extracts were dried over anhydrous Na₂SO₄, filtered and concentrated to dryness in vacuo. Flash chromatography of the crude product (silica gel, CH₂Cl₂/MeOH/NH₃ aq. 150/10/1) gave 245 mg of the cyclized product.

This material (130 mg) was dissolved in TFA (2.5 ml). The reaction mixture was stirred at room temperature for 2 hours, then concentrated in vacuo. A saturated solution of NaHCO₃ was added and the mixture was extracted with ethyl acetate, dried over Na₂SO₄ and concentrated in vacuo. The residue was dissolved in CH₂Cl₂ and HCl (1 M in Et₂O, 2 ml) was added, the volatiles evaporated and the residue triturated with Et₂O to give 130 mg of the title product as a light pink solid:

NMR (¹H, DMSO-d6): δ 9.9 (bs 1H), 7.71 (d, 2H), 7.7-6.64 (m, 2H), 7.67 (t, 1H), 7.09 (d, 1H), 6.99 (d, 2H), 4.56 (d, 2H), 4.39 (bt, 2H), 3.81 (s, 3H), 3.76 (s, 3H), 3.58 (m, 2H), 3.50 (m, 2H), 3.04 (m, 2H), 1.81 (m, 4H), 1.7 (m,1 H), 1.4 (m,1 H). MS (m/z): 423 [MH]⁺.

### EXAMPLE 7

### 1-(4-Methoxyphenyl)-3-[4-methoxy-3-(2-piperidin-1-yl-ethoxy)-phenyl]-pyrrolidin-2-one hydrochloride

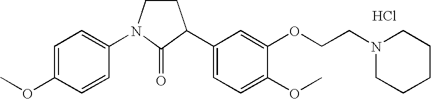

The product was prepared in an analogous way to the one described for Example 3.

NMR (¹H, DMSO-d6): δ 9.87 (bs, 1H), 7.58 (d, 2H), 7.0-6.8 (m, 5H), 4.33 (t, 2H), 3.85 (m, 2H), 3.84 (t, 1H), 3.77 (s, 3H), 3.74 (s, 3H), 3.55 (d, 2H) 3.45 (m, 2H), 3.02 (M, 2H), 2.53 (m, 1H), 2.16 (m,₁ H), 1.85-1.65 (m, 5H), 1.39 (m, 1H). MS (m/z): 425 [MH]⁺.

### EXAMPLE NO. 8

### 1-(3-Chloro-4-methoxy-phenyl)-3-[4-methoxy-3-(2.piperidin-1-yl-ethoxy)-phenyl]-pyrrolidin-2-one hydrochloride

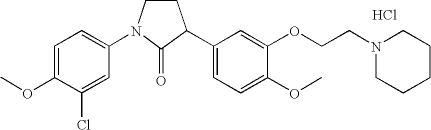

The product was prepared in an analogous way to the one described for Example 3.

NMR (¹H, DMSO-d6): δ 9.75 (bs, 1H), 7.89 (d, 1H), 7.53 (dd, 1H), 7.17 (d, 1H), 6.99 (m, 2H), 6.90 (dd, 1 H), 4.32 (t, 2H), 3.87 (m, 3H), 3.83 (s, 3H), 3.81 (s, 3H), 3.55 (m, 2H), 3.45 (m, 2H), 3.0 (m, 2H), 2.5 (m, 1H), 2.15 (m, 1H), 1.9 (m, 4H), 1.4 (m, 2H). MS (m/z): 459 [MH]⁺, 1Cl.

### EXAMPLE 9

### 1-(3-Chloro-4-methoxy-phenyl)-3-[4-methoxy-3-(2-piperidin-1-yl-ethoxy)-phenyl]-1,5-dihydro-pyrrole-2-one hydrochloride

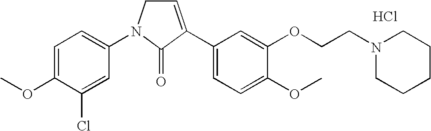

The product was prepared in an analogous way to the one described for Example 6.

NMR (¹H, DMSO-d6): δ 9.76 (bs, 1H), 8.01 (d, 1H), 7.68 (m, 4H), 7.19 (d, 1H), 7.08 (d, 1H), 4.59 (d, 2H), 4.38 (t, 2H), 3.85 (s, 3H), 3.81 (s, 3H), 3.58 (m, 2H), 3.50 (m, 2H), 3.04 (m, 2H), 1.85 (m, 4H), 1.4 (m, 2H). MS (m/z): 457 [MH]⁺, 1Cl.

### EXAMPLE 10

### 1-(3-Fluoro-4-methoxy-phenyl)-3-[4-methoxy-3-(2-piperidin-1-yl-ethoxy)-phenyl]-pyrrolidin-2-one hydrochloride

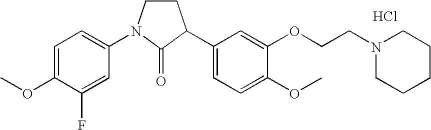

The product was prepared in an analogous way to the one described for Example 3.

NMR (¹H, DMSO-d6): δ 9.82 (bs, 1H), 7.74 (dd, 1H), 7.36 (dd, 1H), 7.17 (t, 1H), 6.99 (m, 2H), 6.90 (dd, 1H), 4.32 (t, 2H), 3.87 (m, 3H), 3.82 (s, 3H), 3.76 (s, 3H), 3.54 (m, 2H), 3.45 (m, 2H), 3.0 (m, 2H), 2.5 (m, 1H), 2.16 (m, 1H), 1.85 (m, 4H), 1.4 (m, 2H). MS (m/z): 443 [MH]⁺.

### EXAMPLE 11

### 1-(3-Fluoro-4-methoxy-phenyl)-3-[4-methoxy-3-(2-piperidin-1-yl-ethoxy)-phenyl]-1,5-dihydro-pyrrole-2-one hydrochloride

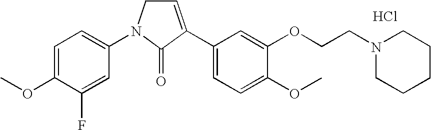

The product was prepared in an analogous way to the one described for Example 6.

NMR (¹H, DMSO-d6): δ 9.87 (bs, 1H), 7.84 (dd, 1H), 7.68 (m, 2H), 7.66 (dd, 1H), 7.51 (m, 1H), 7.21 (t, 1H), 7.08 (d, 1H), 4.58 (d, 2H), 4.39 (t, 2H), 3.84 (s, 3H), 3.81 (s, 3H), 3.57 (bd, 2H), 3.49 (m, 2H), 3.04 (m, 2H), 1.9-1.75 (m, 5H), 1.4 (m, 1H). MS (m/z): 441 [MH]⁺.

### EXAMPLE 12

### 1-(3-Fluoro-phenyl)-3-[4-methoxy-3-(2-piperidin-1-yl-ethoxy)-phenyl]-pyrrolidin-2-one hydrochloride

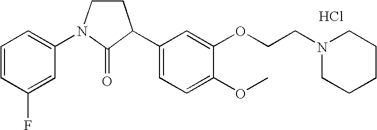

The product was prepared in an analogous way to the one described for Example 3.

NMR (¹H, DMSO-d6): δ 9.75 (bs, 1H), 7.70 (m, 1H), 7.47 (m, 1H), 7.43 (m, 1H), 7.0 (m, 1H), 7.0 (m, 2H), 6.92 (dd, 1H), 4.32 (t, 2H), 3.90 (m, 3H), 3.77 (s, 3H), 3.54 (m, 2H), 3.46 (m, 2H), 3.0 (m, 2H), 2.5-2.2 (m, 2H), 1.82 (m, 4H), 1.4 (m, 2H). MS (m/z): 413 [MH]⁺.

### EXAMPLE 13

### 1-(3-Fluoro-phenyl)-3-[4-methoxy-3-(2-piperidin-1-yl-ethoxy)-phenyl]-1,5-dihydro-pyrrole-2-one hydrochloride

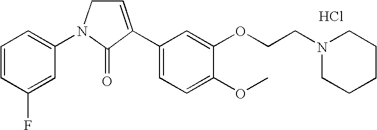

The product was prepared in an analogous way to the one described for Example 6.

NMR (¹H, DMSO-d6): δ 9.9 (bs, 1H), 7.82 (m, 1H), 7.73 (t, 1H), 7.66 (m, 2H), 7.60 (dd, 1H), 7.45 (m, 1H), 7.09 (d, 1H), 6.97 (m, 1 H), 4.62 (d, 2H), 4.39 (t, 2H), 3.82 (s, 3H), 3.57 (m, 2H), 3.48 (m, 2H), 3.02 (m, 2H), 1.9 (m, 4H), 1.4 (m, 2H). MS (m/z): 411 [MH]⁺.

### EXAMPLE 14

### 1-(3,4-Dichloro-phenyl)-5,5-dimethyl-4-hydroxy-3-[4-methoxy-3-(2-piperidin-1-yl-ethoxy)-phenyl]-pyrrol-2-one

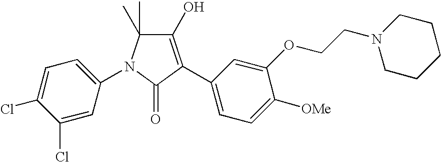

To a solution of 2-((3,4-dichloro-phenyl)-{2-[4-methoxy-3-(2-piperidin-1-yl-ethoxy)-phenyl]-acetyl}-amino)-2-methyl propionic acid methyl ester (0.19 g, 0.35 mmol) in CH₂Cl₂+THF (1.5+1.5 ml) at 0° C. was added NaH (60% in mineral oil, 13.9 mg, 0.35 mmol). The solution was stirred at room temperature for 1.5 hours, cooled at 0° C. and HCl (1M in Et₂O, 0.35 mmol) was added. After stirring the solution at room temperature for 15 minutes, the solvent was removed in vacuo, the crude was dissolved in CH₂Cl₂, filtered and concentrated to dryness in vacuo. After purification by flash chromatography CH₂Cl₂:MeOH=(9:1) the title compound was obtained in 143 mg yield as a yellow solid (82%).

NMR (¹H, DMSO): δ 9.50 (bs,1H), 8.16 (bs, 1H), 7.97 (bd, 1H), 7.83 (d, 1H), 7.53 (d, 1H), 7.34 (dd, 1H), 6.87 (d, 1H), 4.21 (t, 2H), 3.83 (t, 2H), 3.69 (s, 3H), 3.27 (m, 4H), 1.71 (m, 4H), 1.50 (bm, 2H), 1.25 (s, 6H). MS (m/z): 505 [MH]⁺ (2Cl).

### EXAMPLE 15

### 1-(3,4-Dichloro-phenyl)-5,5-dimethyl-4-hydroxy-3-[4-methoxy-3-(2-piperidin-1-yl-ethoxy)-phenyl]-pyrrol-2-one hydrochloride salt

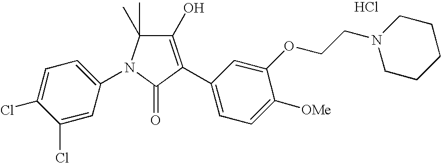

To a solution of 1-(3,4-dichloro-phenyl)-5,5-dimethyl-4-hydroxy-3-[4-methoxy-3-(2-piperidin-1-yl-ethoxy)-phenyl]-pyrrol-2-one in CH₂Cl₂ was added excess HCl (1 M in Et₂O). The resulting mixture was evaporated to dryness and the residue triturated with Et₂O and dried to give the title compound in 5 mg yield as a white solid (66%).

1. A compound of formula (I) or a pharmaceutically acceptable salt thereof:
wherein: 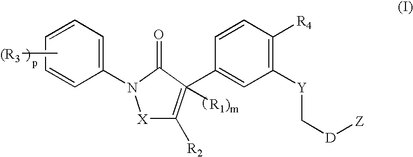
R₁ is hydrogen, fluoro, chloro, hydroxy, C₁₋₆alkyl, C₃₋₇cycloalkyl, C₃₋₇cycloalkyloxy, C₁₋₆alkoxy or haloC₁₋₆alkoxy;
m is 0 when ═ is a double bond and m is 1 when ═ is a single bond;
R₂ is hydrogen, halogen, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group;
X is —(CH₂—CH₂)—, —(CH═CH)—, —(CH₂)₃—, —C(CH₃)₂—, —(CH═CH—CH₂)—, —(CH₂—CH═CH)— or a group —(CHR₅)— wherein R₅ is hydrogen, halogen, hydroxy, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy or C₁₋₆alkylthio;
R₃ is halogen, cyano, C₁₋₆alkyl, C₃₋₇cycloalkyl, C₃₋₇cycloalkyloxy, C₁₋₆alkoxy, C₁₋₆alkylthio, hydroxy, amino, mono- or di-C₁₋₆alkylamino, an N-linked 4 to 7 membered heterocyclic group, nitro, haloC₁₋₆alkyl, haloC₁₋₆alkoxy, aryl, arylC₁₋₆alkyl, arylC₁₋₆alkyloxy, arylC₁₋₆alkylthio or COOR₆, CONR₇R₈ or COR₉ wherein R₆, R₇, R₈ and R₉ are independently hydrogen or C₁₋₆alkyl;
p is 0, 1 or 2 or 3;
R₄ is hydrogen, halogen, hydroxy, cyano, nitro, C₁₋₆alkyl, C₁₋₆alkanoyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group;
Y is oxygen, sulfur, —CH₂— or NR₁₀ wherein R₁₀ is hydrogen or C₁₋₆alkyl;
D is a single bond, —CH₂—, —(CH₂)₂— or —CH═CH—; and
Z is —NR₁₁R₁₂ where R₁₁ and R₁₂ are independently hydrogen or C₁₋₆alkyl, or an optionally substituted N-linked or C-linked 4 to 7 membered heterocyclic group.

2. A compound as claimed in claim 1, wherein X is —CH₂—.

3. A compound as claimed in claim 1, wherein when ═ is a single bond, R₁ is hydrogen, hydroxy or C₁₋₆alkoxy.

4. A compound as claimed in claim 1 having the following formula (Ia): 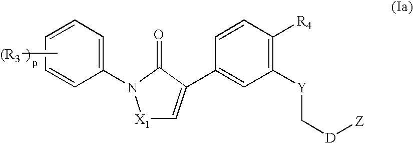
wherein R₃, p, R₄, Y, D, Z, ═ are as defined in claim 1, 2 or 3 and X₁ is —CH₂— or —HC(OH)—.

5. A compound as claimed in claim 1, wherein p is 1 or 2 and R₃ is/are halogen, particularly chloro or fluoro, attached at the 3 or the 3,4-positions of the phenyl ring.

6. A compound as claimed in claim 1, wherein R₄ is C₁₋₆alkoxy (particularly methoxy), OCF₃, halogen or cyano.

7. A compound as claimed in claim 1, wherein D is —CH₂—.

8. A compound as claimed in claim 1, wherein Y is oxygen.

9. A compound as claimed in claim 1, wherein Z is an optionally substituted N-linked 4 to 7 membered heterocycle.

10. A compound as claimed in claim 9, wherein Z is piperidyl.

11. A compound as claimed in claim 1 which is:
1-(3,4-Dichloro-phenyl)-5-hydroxy-3-[4-methoxy-3-(2-piperidin-1-yl-ethoxy)-phenyl]-pyrrolidin-2-one
1-(3,4-Dichloro-phenyl)-3-[4-methoxy-3-(2-piperidin-1-yl-ethoxy)-phenyl]-1,5-dihydro-pyrrole-2-one
1-(3,4-Dichloro-phenyl)-3-[4-methoxy-3-(2-piperidin-1-yl-ethoxy)-phenyl]-pyrrolidin-2-one
1-(3,4-Dichloro-phenyl)-3-[4-methoxy-3-(2-piperidin-1-yl-ethoxy)-phenyl]-3-methyl-pyrrolidin-2-one
1-(4-Methoxyphenyl)-3-[4-methoxy-3-(2-piperidin-1-yl-ethoxy)-phenyl]-1,5-dihydro-pyrrole-2-one
1-(4-Methoxyphenyl)-3-[4-methoxy-3-(2-piperidin-1-yl-ethoxy)-phenyl]-pyrrolidin-2-one
1-(3-Chloro-4-methoxy-phenyl)-3-[4-methoxy-3-(2-piperidin-1-yl-ethoxy)-phenyl]-pyrrolidin-2-one
1-(3-Chloro-4-methoxy-phenyl)-3-[4-methoxy-3-(2-piperidin-1-yl-ethoxy)-phenyl]-1,5-dihydro-pyrrole-2-one
1-(3-Fluoro-4-methoxy-phenyl)-3-[4-methoxy-3-(2-piperidin-1-yl-ethoxy)-phenyl]-pyrrolidin-2-one
1-(3-Fluoro-4-methoxy-phenyl)-3-[4-methoxy-3-(2-piperidin-1-yl-ethoxy)-phenyl]-1,5-dihydro-pyrrole-2-one
1-(3-Fluoro-phenyl)-3-[4-methoxy-3-(2-piperidin-1-yl-ethoxy)-phenyl]-pyrrolidin-2-one
1-(3-Fluoro-phenyl)-3-[4-methoxy-3-(2-piperidin-1-yl-ethoxy)-phenyl]-1,5-dihydro-pyrrole-2-one
1-(3,4-Dichloro-phenyl)-5,5-dimethyl-4-hydroxy-3-[4-methoxy-3-(2-piperidin-1-yl-ethoxy)-phenyl]-pyrrol-2-one
1-(3,4-Dichloro-phenyl)-5,5-dimethyl-4-hydroxy-3-[4-methoxy-3-(2-piperidin-1-yl-ethoxy)-phenyl]-pyrrol-2-one or a pharmaceutically acceptable salt thereof.

12. A process for the preparation of a compound according to claim 1, which process comprises:
(a) reacting a compound of formula (II): 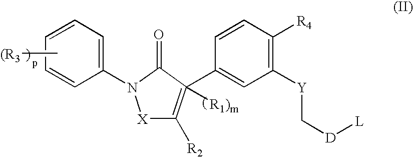
wherein R₁, R₂, R₃, R₄, m, p, X, ═, Y and D are as defined for formula (I), and L is a leaving group, with a compound of formula (III):
Z-H (III)
wherein Z is as defined for formula (I); or 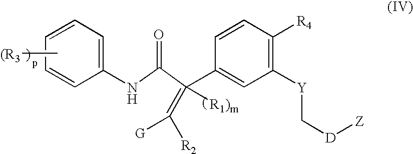
(b) cyclising a compound of formula (IV):
wherein R₁, R₂, m, R₃, p, R4, Y, D, Z and ═ are as defined for formula (I) and G is a group —X═CH₂, wherein X is as defined for formula (I), dehydrogenated as required;
and thereafter, for either process (a) or process (b), optionally followed by:
removing any protecting groups; and/or
converting a compound of formula (I) into another compound of formula (I); and/or
forming a pharmaceutically acceptable salt.

13. A pharmaceutical composition comprising a compound according to claim 1, and a pharmaceutically acceptable carrier or excipient.

14. (canceled)

15. A compound according to claim 1 for use as a therapeutic substance.

16. A compound according to claim 1 for use in the treatment of a CNS disorder.

17. A compound according to claim 1 for use in the treatment of depression or anxiety.

18. A method of treatment of CNS disorder in a mammal including a human, which comprises administering to the sufferer a therapeutically safe and effective amount of a compound according to claim 1.

19. A method of treatment of depression or anxiety in a mammal including a human, which comprises administering to the sufferer a therapeutically safe and effective amount of a compound according to claim 1.

20. (canceled)

21. (canceled)

22. A process comprising mixing a compound a compound according to claim 1 and a pharmaceutically acceptable carrier or excipient.
